(19) United States
(12) Patent Application Publication
(10) Pub. No.: US 2010/0094233 A1
(43) Pub. Date: Apr. 15, 2010

(54) Apparatus for retaining & disposing bodily fluids

(76) Inventor: Paul Ashworth, Arroyo Grande, CA (US)

Correspondence Address: LAW OFFICES OF ANDREW SCHROEDER, P.O. Box 6731, Santa Maria, CA, 93454, US

(21) Appl. No.: 12/288,004

(22) Filed: Oct. 14, 2008

(51) Int. Cl. A61M 1/00

(52) U.S. Cl. 604/317

(57) ABSTRACT

An Apparatus for Capturing Bodily Fluids and Disposing the Bodily Fluids to another location includes a Funnel and a Conduit. The Funnel comprises a capture distal end and a mating distal end. The Conduit comprises an elongate tube. The elongate tube comprises an anterior distal end and a posterior distal end. In some preferred embodiments the posterior end of the conduit is threaded. The anterior distal end of the Conduit is in communication with the capture distal end of the Funnel.

### FIELD OF THE INVENTION

The present invention is in the area of healthcare and more particularly pertains to a retainer and conduit for use with various bodily fluids.

### BACKGROUND OF THE INVENTION

One of the problems plaguing many infirm or sick people is the capture, retention, and disposal of various bodily fluids. The two most common challenges which face many sick people are vomiting and urination. Aside from the odor, sight, smell and other attendant effects, various health and safety issues are brought to bear. For example, urine is acidic and prolonged exposure of urine to human skin is unhealthy. Likewise, discharge from vomiting may harbor various germs, or other pathogens which may constitute a hazard to the sick person as well as others in the vicinity.

One particular example which besets many sick people is urination in the evening. Many sick people are confronted with severe difficulty urinating into a toilet due to shaking, weakness, or other conditions. As a result, many people may urinate onto the floor creating a mess. And likewise, the same issue arises when these people go to the bathroom to vomit.

In particular, when a sick person attempts to vomit, they must bend down onto their knees and place their mouth near an unsanitary toilet bowl. And in the process of kneeling down near the toilet bowl, the person may collapse due to weakness or other conditions which plague them. Furthermore, their weakened condition may make it extremely difficult, and dangerous in some circumstances if they must stand up. Oftentimes, they may not have the physical strength to stand up. Or in other cases, blood may rush from their brain downwards creating a chance for fainting.

Another closely related issue is found when a sick person is confined to a bed. Many times hospitals and other health care facilities use bed-adapted urinals which are used to collect urine. The problem with these bed-adapted urinals is that they are uncomfortable, and it may be necessary to have another person personally remove it away from the bed. However, a person who is unattended may not be able to have the bed-adapted urinals removed which may complicate their weakened condition further. Moreover, many bed-ridden people have difficulty vomiting into a trash can or similar container near their bed.

For the reasons given above, what is clearly needed in the marketplace is an apparatus, which may enable a person to use the toilet while standing up. This apparatus may use the toilet as a receptacle for vomit and urination. This apparatus may be articulable to move up and down, and side to side in order to accommodate people of various heights.

Moreover, another apparatus needed in the art is a system adapted for use with bed-ridden patients who may not have the strength to go to the bathroom. The apparatus should enable a person to easily vomit or urinate into a funnel and dispose of the fluids or substances into another location where the stench of the fluids is not as problematic. The funnel should be articulable and within convenient reach of the person.

In addition, another apparatus needed in the art is a portable system which may be used in confined spaces such as cars or other enclosed spaces.

### SUMMARY OF THE INVENTION

It is an object of the present invention to enable a user to vomit or urinate into a toilet without creating a mess. Large funnels may be used and disposed close enough to a person to ensure that the urine is completely disposed within the toilet.

It is also an object of the present invention to enable a user to vomit into a toilet without crouching on their knees so that their mouth is near a toilet. By enabling a user to vomit while standing up in an erect position, the user may avoid injuring themselves either by falling as they kneel near the bowl and as they get up after they have used the toilet. In addition, the user will not have to worsen their condition further by placing their mouth in near contact with an unhygienic toilet bowl.

It is also an object of the present invention to provide a convenient and portable system for a person who may need to either vomit or urinate in a closed space such as a vehicle.

It is also an object of the present invention to provide a system adapted for use with beds. By disposing a funnel near the user which is articulable, a person will not have to go to the bathroom to urinate and vomit. Moreover, the use of an uncomfortable apparatus for collecting urine, or vomit may also be avoided.

### BRIEF DESCRIPTION OF THE DRAWING FIGURES

FIG. 1A is a front elevation view of a preferred embodiment of the present invention.

FIG. 1B is a front elevation view of a preferred embodiment of the present invention.

FIG. 2 is a front elevation view of a preferred embodiment of the present invention.

FIG. 3 is a front elevation view of a preferred embodiment of the present invention.

FIG. 4 is a front elevation view of a preferred embodiment of the present invention.

FIG. 5 is a plan view of a preferred embodiment of the present invention.

FIG. 6 is a plan view of a preferred embodiment of the present invention.

FIG. 7 is a front elevation view of a preferred embodiment of the present invention.

FIG. 8 is a plan view of a preferred embodiment of the present invention.

FIG. 9 is a plan view of a preferred embodiment of the present invention.

FIG. 10 is a plan view of a preferred embodiment of the present invention.

FIG. 11 is a plan view of a preferred embodiment of the present invention.

FIG. 12A is a side elevation view of a preferred embodiment of the present invention.

FIG. 12B is a cross-section view of a preferred embodiment of the present invention.

FIG. 13 is a side elevation elevation view of a preferred embodiment of the present invention.

FIG. 14 is a plan elevation view of a preferred embodiment of the present invention.

FIG. 15 is a front elevation view of a preferred embodiment of the present invention.

FIG. 16 is a side elevation view of a preferred embodiment of the present invention.

FIG. 17 is a side elevation view of a preferred embodiment of the present invention.

FIG. 18 is a side elevation view of a preferred embodiment of the present invention.

FIG. 19 is a side elevation view of a preferred embodiment of the present invention.

FIG. 20 is a side elevation view of a preferred embodiment of the present invention.

FIG. 21 is a rear elevation view of a preferred embodiment of the present invention.

FIG. 22 is a front elevation view of a preferred embodiment of the present invention.

FIG. 23 is a side elevation view of a preferred embodiment of the present invention.

FIG. 24 is a side elevation view of a preferred embodiment of the present invention.

FIG. 25 is a side elevation view of a preferred embodiment of the present invention.

FIG. 26 is a side elevation view of a preferred embodiment of the present invention.

FIG. 27 is a side elevation view of a preferred embodiment of the present invention.

FIG. 28 is a side elevation view of a preferred embodiment of the present invention.

FIG. 29 is a side elevation view of a preferred embodiment of the present invention.

FIG. 30 is a side elevation view of a preferred embodiment of the present invention.

FIG. 31 is a side elevation view of a preferred embodiment of the present invention.

FIG. 32 is a plan view of a preferred embodiment of the present invention.

FIG. 33 is a plan view of a preferred embodiment of the present invention.

FIG. 34 is a plan view of a preferred embodiment of the present invention.

FIG. 35 is a plan view of a preferred embodiment of the present invention.

FIG. 36 is a rear elevation view of a preferred embodiment of the present invention.

FIG. 37 is a side elevation view of a preferred embodiment of the present invention.

FIG. 38 is a side elevation view of a preferred embodiment of the present invention.

FIG. 39 is a plan view of a preferred embodiment of the present invention.

### DESCRIPTION OF PREFERRED EMBODIMENTS

According to a preferred embodiment of the present invention, a unique apparatus is used to capture, and dispose of vomit, urine, or other bodily fluids and/or substances. The invention is also used to dispose these fluids and substances to another location such as a container. The invention is described in enabling detail below.

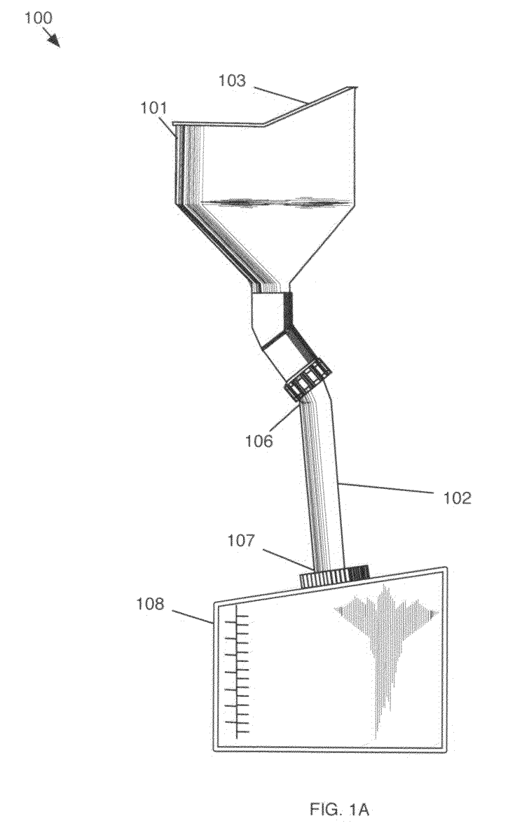

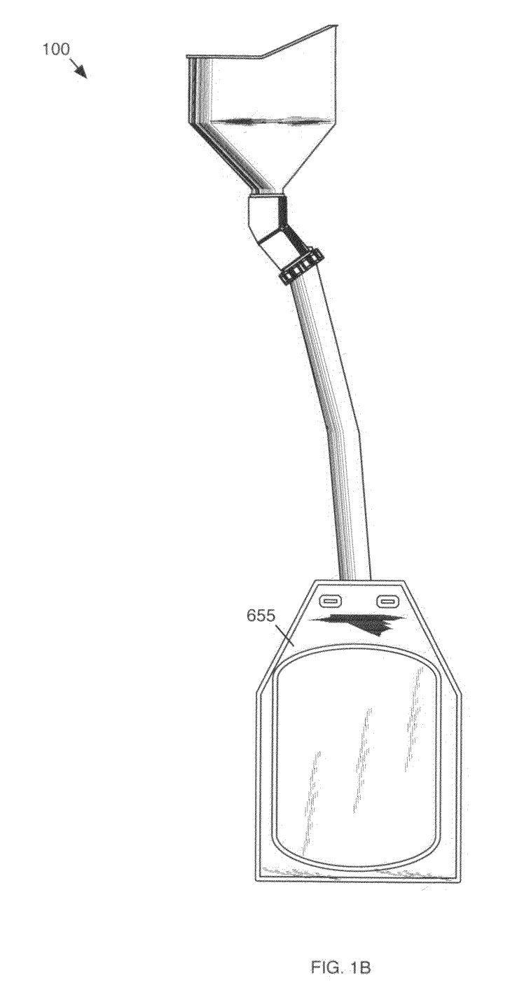

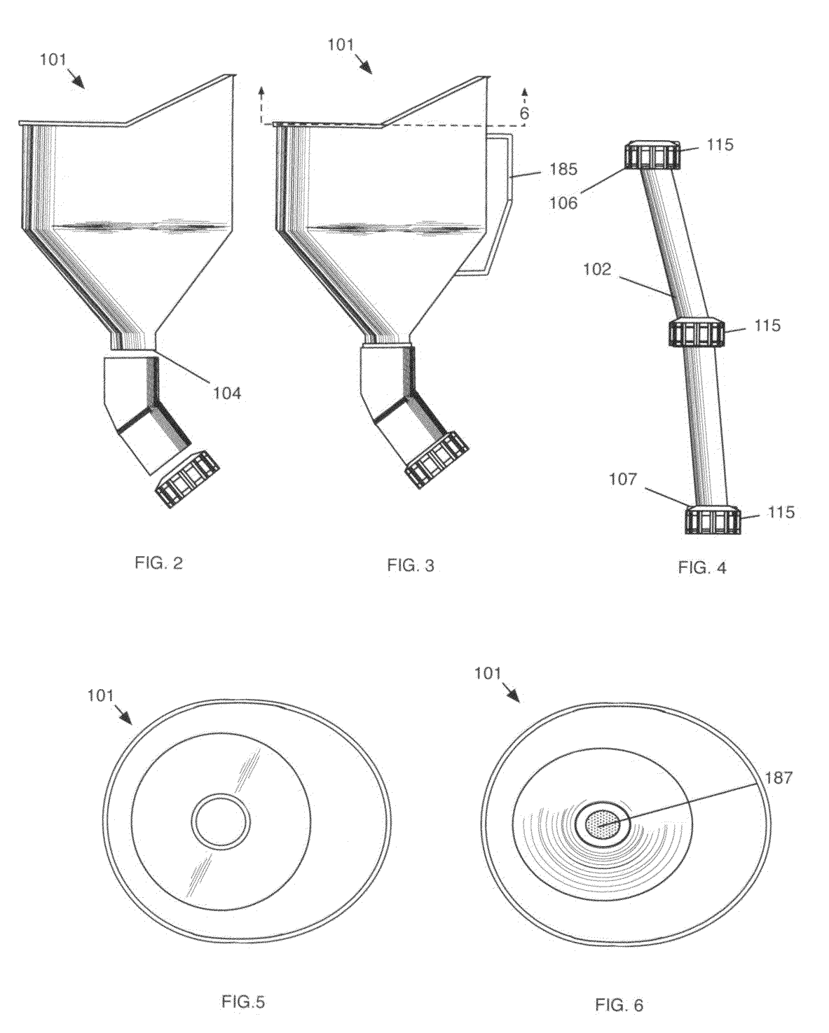

FIG. 1A illustrates a preferred embodiment of the present invention. An Apparatus for Capturing Bodily Fluids and Disposing the Bodily Fluids to another location 100 includes a Funnel 101 and a Conduit 102.

The Funnel 101 comprises a capture distal end 103 and a mating distal end 104. In some preferred embodiments, the Funnel 101 may further incorporate a filter 187, which is disposed near the orifice. The Funnel 101 may also incorporate a handle 185. The handle 185 may become expedient for the purpose of turning or articulating the Funnel 101 away from the user as illustrated in FIGS. 13-14.

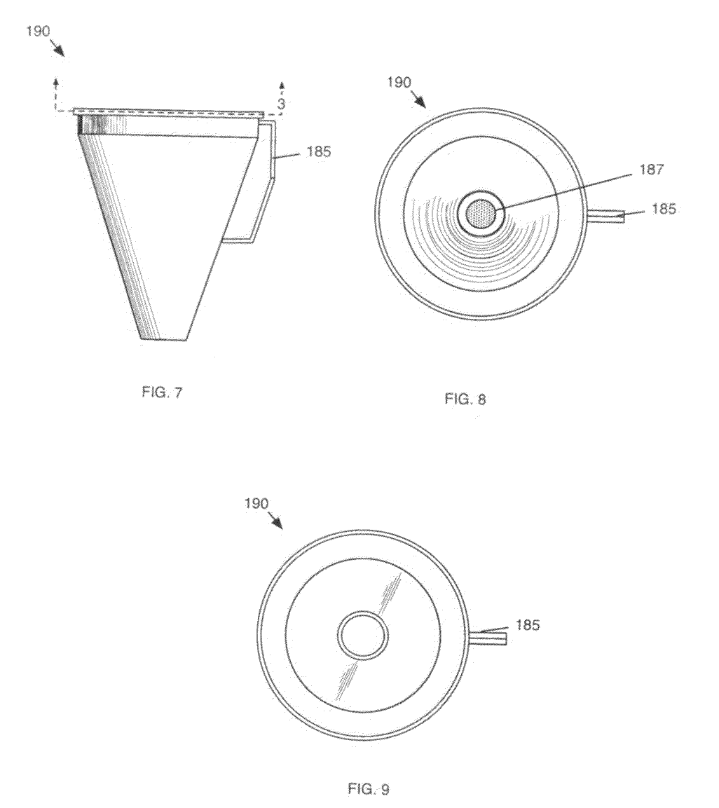

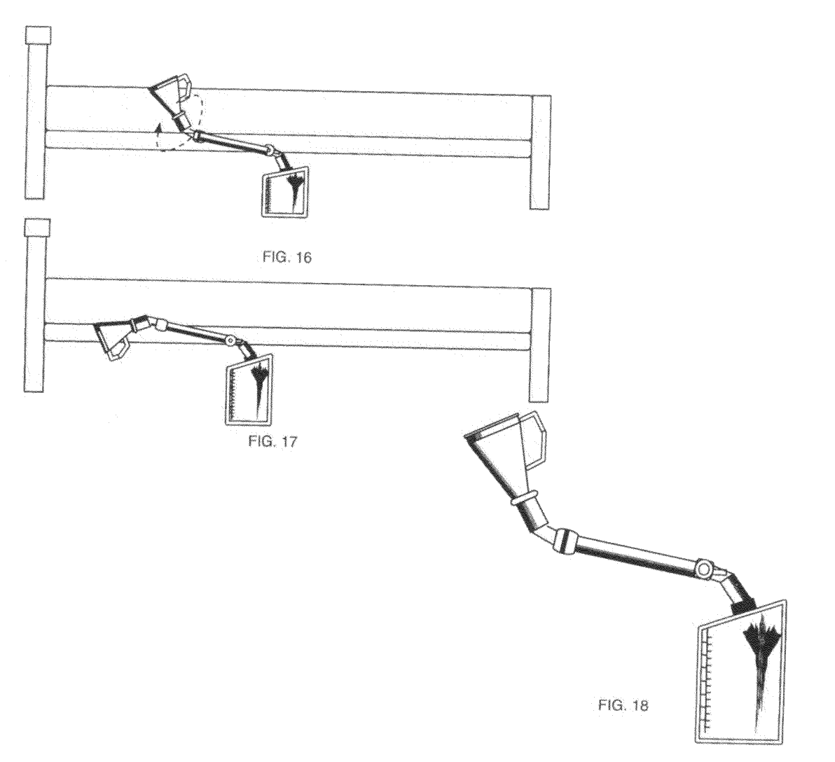

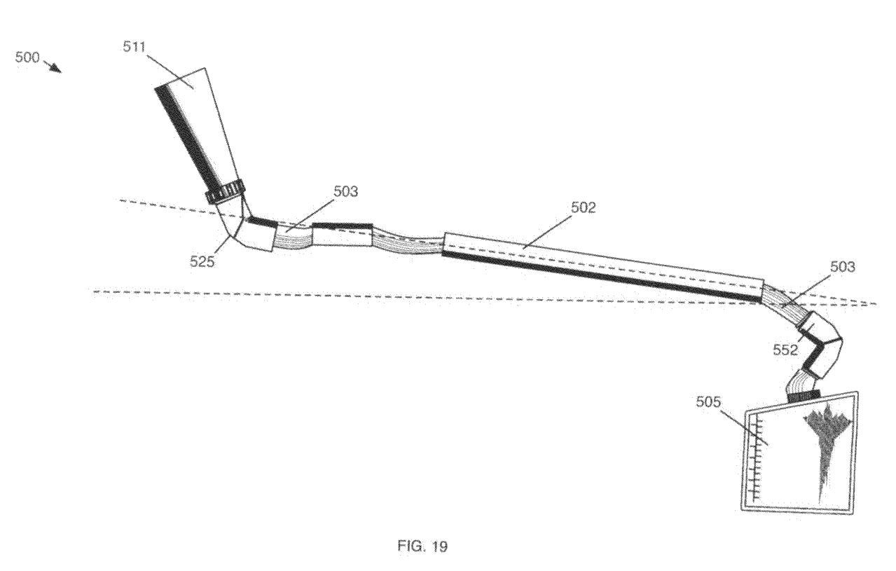

FIGS. 7-9 illustrate another version of a funnel. The Alternate Funnel 190 may be used in lieu of the Funnel 101. FIG. 19 illustrates that an Elongated Funnel 511 may also be used with preferred embodiments of the present invention. The Funnel 101, the Elongated Funnel 511, or the Alternate Funnel 190 may be comprised of plastic, metal, or other suitable materials.

The Conduit 102 comprises an elongate tube and various attachment pieces 115. The attachment pieces 115 may include elbows, three-way junctions, etc. FIGS. 13-14 illustrate that the attachment pieces 115 may also allow for articulation of the Conduit 102 or Funnel 101 as desirable. The elongate tube comprises an anterior distal end 106 and a posterior distal end 107. In some preferred embodiments the posterior end of the Conduit 102 is threaded. The anterior distal end 106 of the Conduit 102 is in communication with the capture distal end 103 of the Funnel 101.

In some preferred embodiments, the Apparatus for Capturing Bodily Fluids and Disposing the Bodily Fluids to another location 100 may also include a container 108. The container 108 is in communication with the posterior distal end 107 of the Conduit 102. FIG. 1B illustrates that the container may be a plastic, collapsible bag 655 in some preferred embodiments.

In some preferred embodiments, the attachment point of the container 108 may be complimentarily threaded to the posterior distal end 107 of the elongate tube 105. The Container 108 may take a panoply of different forms, sizes, shapes, etc. In addition, the Container 108 may be disposable in some preferred embodiments whereas in other preferred embodiments the Container 108 may be re-usable. Re-usable Container 108 may be comprised of durable plastics, glass, or similar materials.

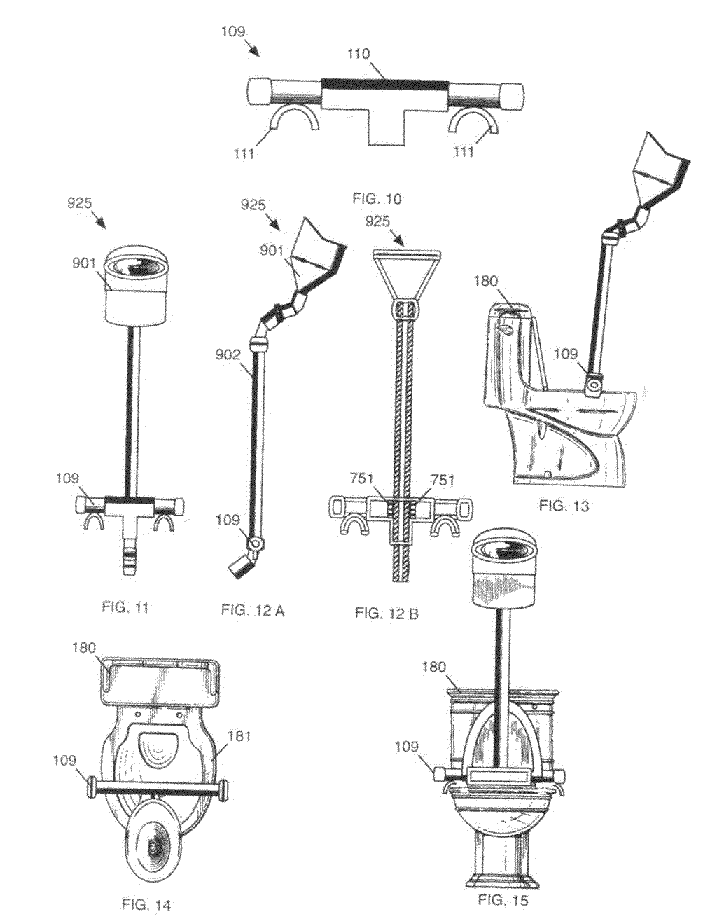

FIGS. 10-15 illustrate another preferred embodiment of the present invention. A Vomiting Apparatus 925 incorporates a Receptacle Funnel 901, a Funneling Conduit 902, and a toilet adapter assembly 109. The toilet adapter assembly 109 is comprised of a cross-bar 110 and at least one attachment member 111. The attachment member 111 is pliable and resilient. The attachment member 111 is sized to mate with the rim 181 of a toilet bowl 180. In some preferred embodiments, the attachment member 111 is made of plastic. In other preferred embodiments, the attachment member 111 may be made of rubber or other suitable materials. FIG. 12 B illustrates that part of the cross-bar 110 may be blocked off with partitions 751 in some preferred embodiments.

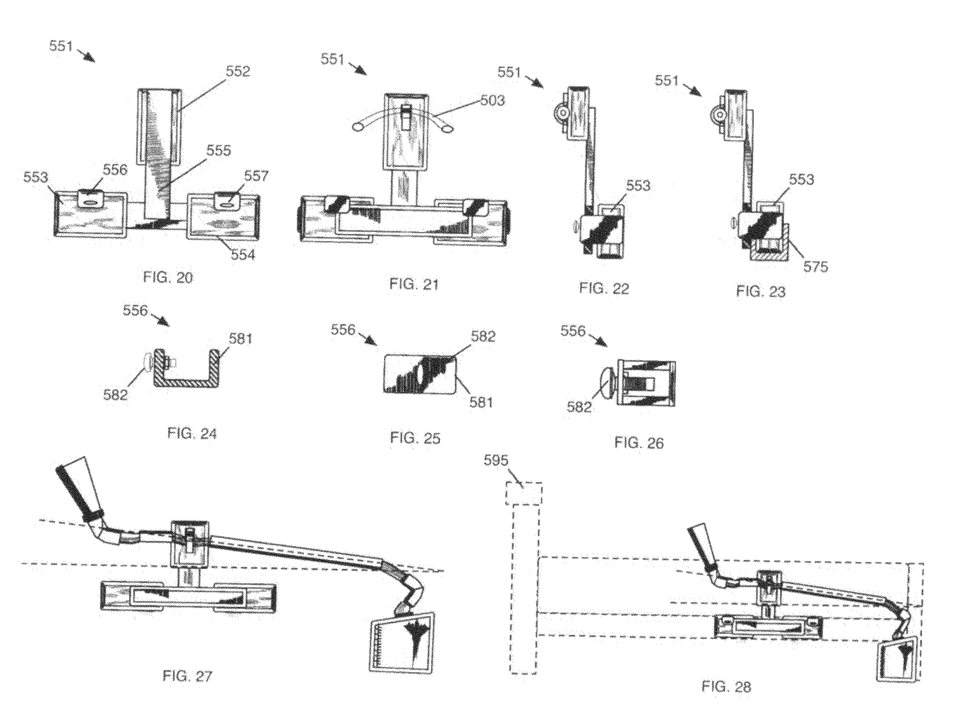

FIG. 20 illustrates another preferred embodiment of the present invention. A Bodily Fluid Collection and Retaining Apparatus 500 includes a Collector 511, at least one Sleeve 502, 504, a Hose 503, and a Retaining Container 505. In some preferred embodiments, the Bodily Fluid Collection and Retaining Apparatus 500 may further comprise at least one Elbow 525.

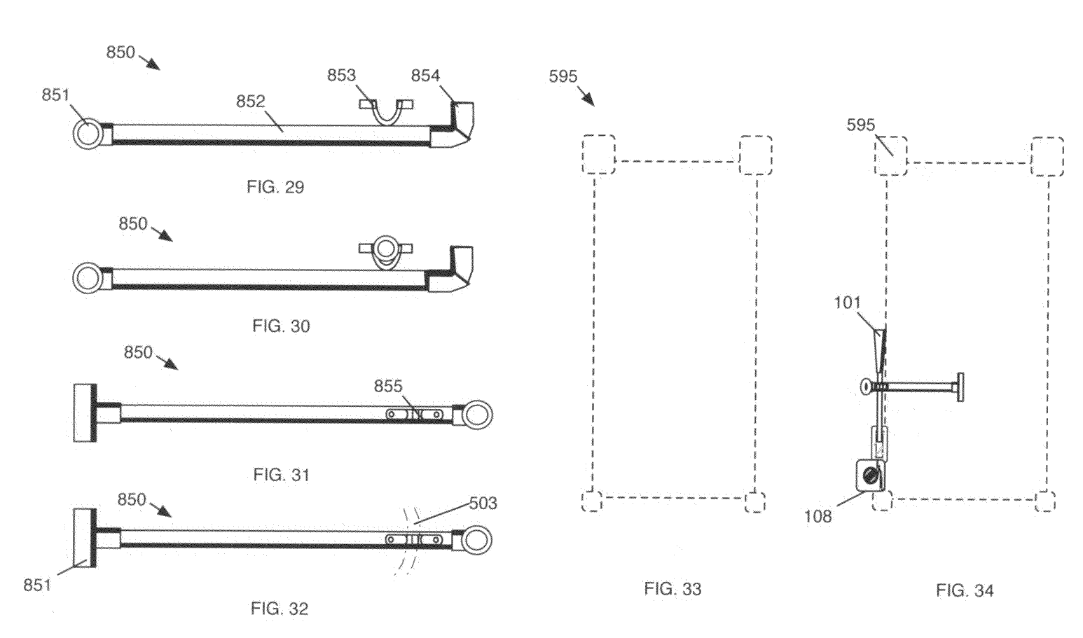

FIGS. 20-28 illustrate that the Apparatus for Capturing Bodily Fluids and Disposing the Bodily Fluids to another location 100 may further incorporate a bed adapter assembly 551. The bed adapter assembly 551 is comprised of three planar members 552, 553, 554 and a T-Bar 555. The planar members 553, 554 are used to sandwich a side 575 of a bed frame 595 or similar structure in order to secure the Conduit 102 or elongate tube 503 to the bed frame 595. The three planar members 553, 554 are sandwiched by turning the fastener 582 of the vice apparatus 556. In some preferred embodiments, the fastener 582 may be a wing nut. In other preferred embodiments the fastener 582 may be a screw, bolt or similar fastener 582. It should be noted here that the Conduit 102 should be disposed at an angle to allow for the human fluids to flow into the Container 108 as illustrated in FIGS. 28-29.

FIGS. 30-35 illustrate that the Bodily Fluid Collection and Retaining Apparatus 500 may further incorporate a Positioning Apparatus 850 for the purpose of securing the Conduit 102 or elongate tube 503 securely. The Positioning Apparatus 850 is comprised of a first member 851, a middle member 852, and an end member 854. In some preferred embodiments the Positioning Apparatus 850 may further incorporate a clamp assembly 853. The elongate tube 503 or Conduit 102 is disposed in the grooves 855 of the clamp assembly 853.

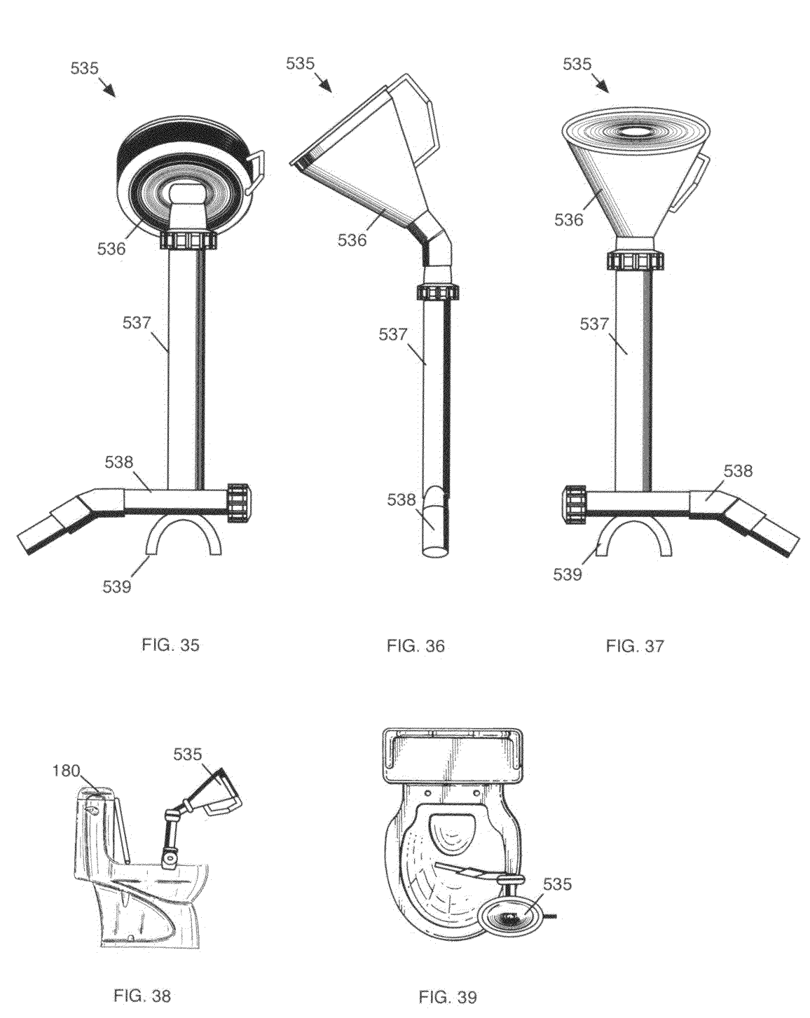

FIGS. 35-39 illustrate another preferred embodiment of the present invention. A Urinating Apparatus 535 comprises a Receiver 536, a Tube 537, a Bottom Member 538, and a Rim Engaging Member 539. The Receiver 536 is in communication with the Tube 537. The Tube 537 is in communication with the Bottom Member 538. And the Bottom Member 538 is in communication with the Rim Engaging Member 539. The Rim Engaging Member 539 is used for the purpose of straddling the rim of a toilet 180 as illustrated in FIGS. 38-39.

It will be apparent to the skilled artisan that there are numerous changes that may be made in embodiments described herein without departing from the spirit and scope of the invention. As such, the invention taught herein by specific examples is limited only by the scope of the claims that follow.

What is claimed is:

1. An Apparatus for Retaining Bodily Fluids and Disposing the Bodily Fluids to another location comprises:
a Funnel and a Conduit;
the Funnel comprises a capture distal end and a mating distal end;
the Conduit comprises an elongate tube;
the elongate tube comprises an anterior distal end and a posterior distal end;
the anterior distal end of the Conduit is in communication with the capture distal end of the Funnel.

2. The Apparatus for Retaining Bodily Fluids and Disposing the Bodily Fluids to another location of claim 1 further comprising a container; the container is in communication with the posterior distal end of the Conduit.

3. The Apparatus for Retaining Bodily Fluids and Disposing the Bodily Fluids to another location of claim 1 wherein the posterior end of the conduit is threaded.

4. The Apparatus for Retaining Bodily Fluids and Disposing the Bodily Fluids to another location of claim 1 wherein the posterior distal end of the Conduit is complimentarily threaded with the posterior end of the Conduit.

5. The Apparatus for Retaining Bodily Fluids and Disposing the Bodily Fluids to another location of claim 1 wherein the conduit further comprises a toilet adapter assembly; the toilet adapter assembly is comprised of a cross-bar and at least one attachment member.

6. The Apparatus for Retaining Bodily Fluids and Disposing the Bodily Fluids to another location of claim 1 further including a hose.

7. The Apparatus for Retaining Bodily Fluids and Disposing the Bodily Fluids to another location of claim 1 further comprising a bed adapter assembly; the bed adapter assembly is comprised of two planar members and a fastener.
